(19) United States
(12) Patent Application Publication
(10) Pub. No.: US 2024/0123150 A1
(43) Pub. Date: Apr. 18, 2024

(54) AUTOINJECTOR WITH AN ELECTRONIC MODULE

(71) Applicant: Ypsomed AG, Burgdorf (CH)

(72) Inventors: Leos Urbanek, Bern (CH); Markus Tschirren, Burgdorf (CH); Martin Brügger, Bolligen (CH); Gabriel Kalbermatter, Burgdorf (CH); Simon Martin Bosshard, Hindelbank (CH); Dominik Zumstein, Bern (CH); Christian Schrul, Oberburg (CH); Mario Bernhard, Burgdorf (CH); Patrick Hostettler, Hasle (CH); Florian Kühni, Bern (CH)

(21) Appl. No.: 18/396,968

(22) Filed: Dec. 27, 2023

Related U.S. Application Data
(63) Continuation of application No. PCT/EP2022/068113, filed on Jun. 30, 2022.

(30) Foreign Application Priority Data
Jun. 30, 2021 (CH) 070001/2021

(51) Int. Cl. A61M 5/20; A61M 5/315; A61M 5/32

(52) U.S. Cl. CPC A61M 5/2033; A61M 5/3157; A61M 5/3213; A61M 2205/50; A61M 2205/58

(57) ABSTRACT

An autoinjector for dispensing a liquid product, particularly a drug, comprising:
an electronic module with a sensor configured to measure the axial movement of a signal element (11) from a position at the start of the dispensing, wherein the signal element (11) is slaved in the dispensing direction, to a position at the end of the dispensing, wherein the signal element (11) strikes the signal stop (12a).

### CROSS-REFERENCE TO RELATED APPLICATIONS

This application is a continuation of International Application No. PCT/EP2022/068113 filed on Jun. 30, 2022 entitled “AUTOINJECTOR WITH AN ELECTRONICS MODULE”, which is claims priority to Swiss Application No. 070001/2021 filed on Jun. 30, 2021 entitled “AUTOINJECTOR WITH AN ELECTRONICS MODULE”, each of which is incorporated by reference herein, in the entirety and for all purposes.

### TECHNICAL FIELD

The invention relates to an autoinjector, often also referred to as an auto-injection device, with which a product contained in a product container can be automatically dispensed after triggering. The liquid product is particularly a drug. Furthermore, the invention relates to an autoinjector comprising an electronic module with a sensor for measuring the axial movement of a signaling apparatus from a position at the start of dispensing to a position at the end of dispensing. This electronic module can be used to detect the start and/or end of injection.

### BACKGROUND

Auto-injectors are known from the prior art, such as from EP2742962A2, to easily and safely deliver an injection with a preloaded spring.

It is also desirable for the patient or medical staff if the start and/or end of distribution of such an injection can be registered or sensed. Thus, the effectiveness of a therapy can be determined by determining the timing of each injection. It is also desirable to automate this logging and dose monitoring.

For autoinjectors, it is further desirable that automatic logging and dose monitoring is simple and safe and that costs are also kept low.

WO2020/016313A1 discloses an autoinjector which has an electronic module for logging and monitoring the dosage of the injection.

### SUMMARY

It is an object of the invention to provide an alternative autoinjector which has an electronic module for logging and monitoring the dosage of the injection.

The object is achieved with the autoinjector according to claim 1. Advantageous further developments result from the dependent claims, the description and the figures.

The autoinjector according to the invention has a housing and a product container arranged in the housing. The product container can particularly be a syringe, which has a syringe body at the distal end of which an injection needle is firmly arranged. The cylindrical syringe body surrounds a plunger which is displaceable with respect to the syringe body and is displaced towards the distal end for product dispensing, whereby the liquid product, particularly drug, arranged between the plunger and the injection needle is dispensed from the product container by the injection needle. The syringe body may have a flange, which may also be referred to as a finger flange, at its proximal end, i.e. the rear end or the end opposite the injection needle. A syringe designed in this way is available as a standard syringe, so there is no need to develop a specially adapted syringe for the autoinjector. The plunger seals against the inside diameter of the syringe body.

The housing may be elongated and forms the longitudinal axis of the autoinjector. The housing may be sleeve-shaped and/or cylindrical, in particular circular cylindrical. The product container is arranged in the housing. For example, the container can be displaceably arranged in the housing, i.e. can be displaced in the distal direction relative to the housing for automatic insertion, so that the needle tip emerges from an opening at the distal end of the autoinjector and can be automatically inserted into the patient. Optionally, the needle tip of such a device can be moved into the distal end of the device after the product has been dispensed, in particular the product container can be moved relative to the housing in the proximal direction.

In some embodiments, the product container is accommodated in the housing in such a manner that it cannot be displaced along the longitudinal axis, in particular by means of a product container holder or syringe holder, which holds the product container in an axially fixed manner and is connected to the housing in an axially fixed manner, in particular latched. The needle tip protrudes over the distal end of the housing in the distal direction. This allows the needle to be inserted into the puncture site by means of a movement of the housing towards the patient. A needle protection sleeve is provided, which forms the distal end of the autoinjector and has an opening for the injection needle, wherein the needle can pass through the opening. The needle protection sleeve can be arranged in its initial position in relation to the needle tip so that the needle protection sleeve stands distally over the needle tip or so that the needle tip stands distally over the distal end of the needle protection sleeve. The needle protection sleeve is displaceable relative to the housing from its initial position in the proximal direction by an actuating stroke into an actuated position, particularly displaceable into the housing, so that the needle protrudes from the distal end or through the opening of the needle protection sleeve or protrudes further. The needle protection sleeve can be displaced by one needle protection stroke from the actuated position relative to the housing in the distal direction to a needle protection position in which the distal end of the needle protection sleeve is distally above the needle tip in order to prevent a risk of injury from an exposed needle tip after the device has been used or after the product has been dispensed. The needle shielding sleeve can, for example, be moved against the force of a spring, which can be referred to as needle shielding spring, in the proximal direction, wherein the spring, which is, for example, the second spring described below or a spring separate therefrom, can move the needle shielding sleeve from the actuated position in the distal direction, i.e., into the needle shielding position. The autoinjector can have a locking member, which, for example, is resiliently arranged and locks the needle shielding sleeve in its needle shielding position, in particular with respect to the housing, and blocks a pushing back of the needle shielding sleeve in the proximal direction or into the housing. The locking element locks the needle protection sleeve at least in such a manner that the needle cannot protrude from the distal end of the needle protection sleeve. The needle shielding sleeve can, for example, be moved from the needle shielding position only to such an extent in the proximal direction that the needle tip does not protrude from the distal end of the needle shielding sleeve.

The autoinjector further comprises a drive element which acts on the plunger at least during the discharge of the product, in particular rests against the plunger, and a first spring which acts on the drive element such as, for example, bracing in particular with its distal end against the drive element. The drive element can, for example, be sleeve-shaped. Further, the propulsion element may comprise one or more ribs arranged, for example, in the area of the distal end of the propulsion element. The distal end of the first spring can brace against the rib. The rib can extend in the proximal direction. The rib can be provided in the interior of the drive element. The rib of the drive element can be provided, for example, on an inner side of the drive element. The first spring may be arranged within the sleeve-shaped drive element. The rib of the drive element can serve to adjust the spring tension of the first spring. For example, a longer designed rib can generate a higher spring preload. The rib can be designed in such a manner that the spring tension of the first spring is or is preloaded in such a way that the liquid product, particularly the totality of the drug in the product container, can be poured out. The length of the ribs or the rib can determine a preload of the first spring such that it can discharge the product from the product container by displacement of the propulsion element by one discharge stroke. The rib can also serve to reinforce the drive element, in particular the part of the drive element against which the first spring braces. This makes it possible to prevent the drive element from being damaged, in particular fractured by the spring force of the first spring. In an alternative embodiment, different first springs with different spring tensions can be or can be used inside the propulsion element as required. By selecting a corresponding first spring, the spring tension can be selected in such a manner that the liquid product, particularly the totality of the drug in the product container, can be poured out. The first spring may be a helical spring which acts as a compression spring and may be formed of metal. The first spring is preloaded to such an extent, particularly in the delivery state of the autoinjector, that it or the energy stored in it is sufficient to substantially completely discharge the product from the product container by displacing the propulsion element by one discharge stroke. The displacement of the propulsion element by the discharge stroke also displaces the piston. If there is a distance between the piston and the propulsion element in the as-delivered state, the discharge stroke of the piston is smaller than the discharge stroke of the propulsion element, which may be as the piston remains unloaded until it is used, thus avoiding unwanted premature product discharge. In principle, however, it is also possible for the propulsion element to be in contact with the piston in the as-delivered state and not only when the product is discharged. If the propulsion element is already in contact with the piston in the as-delivered state, the discharge stroke of the piston corresponds to the discharge stroke of the propulsion element. The proximal end of the first spring, which can also be referred to as a discharge spring due to its function, can brace against the housing or an element fixed to the housing, in particular also an element that is axially fixed to the housing only temporarily.

According to the invention, the autoinjector has a signal member, a signal stop and a second spring. The second spring can exert a spring force acting on the signal member counter to the discharge direction or in the proximal direction. In particular, the second spring can brace with its proximal end against the signal member, for example.

The second spring can be, for example, a helical spring acting as a compression spring, which braces with its proximal end against the signal member. The second spring may be made of metal. The second spring can brace with its distal end, for example, against the housing or an element fixed to the housing. The distal end of the second spring braces against the needle shielding sleeve or an element which, in particular when the needle shielding sleeve is moved relative to the housing, is moved with the needle shielding sleeve. For example, the element can be a switching module or a switching sleeve as described below. The element can in particular be arranged kinematically and/or geometrically between the needle shielding sleeve and the distal end of the second spring. The advantage here is that the needle shielding sleeve is movable from its actuated position into the needle shielding position by means of the second spring. The spring can thus perform a dual function, because it additionally exerts the aforementioned force on the signal member.

In particular in the delivery state or during a first partial stroke of the discharge stroke of the drive element, the signal member can be coupled to the drive element in an axially fixed manner, so that the signal member is movable with the drive element along the longitudinal axis and in particular in the distal direction relative to the housing. The axially fixed coupling to the drive element causes the signal member to be entrained or slaved during the displacement of the drive element in the discharge direction, in particular during the execution of the first partial stroke of the discharge stroke, and causes the second spring to be tensioned or biased. During a second partial stroke of the discharge stroke, it may be that the axially fixed coupling between the signal member and the drive element is released. The axially fixed coupling between the signal member and the drive element is thus releasable. If the axially fixed coupling between the signal element and the propulsion element is released—and particularly if there are no further couplings between the signal element and a further element, as described below—the signal element can be accelerated against the direction of discharge and relative to the propulsion element and/or the housing by means of the second, preloaded spring. Due to the entrainment of the signal member by the drive element by the first partial stroke, a distance, which extends, for example, along the longitudinal axis, is formed between the signal stop and the signal member, said distance in particular corresponding to the first partial stroke. The second spring can accelerate the signal member at this distance, whereby the signal member impinges on the signal stop at a speed such that a pulse is output to the signal member, which pulse generates an acoustic (audible) and/or tactile (feelable) signal. In alternative embodiments, the signal element can hit the signal stop at a speed without a pulse, so that no acoustic (audible) and/or tactile (perceptible) signal is generated.

The signal stop can be formed by the housing or an element which is at least axially fixed, or can also connected to the housing in a rotationally fixed manner. For example, this element can be a closure cap at the proximal end of the housing and/or form the proximal end of the autoinjector. The sealing cap can be designed in one or more parts. The closure cap can be connected to the housing in a positive-locking manner, alternatively in a friction-locking or integral manner The element is locked to the housing. A separate closure cap has the advantage that the installation of the device is facilitated, wherein for the installation at least a portion of the components is introduced into the housing via the proximal end, which is then sealed off with the closure cap. The cap can form a resonating body if the signal stop is arranged on the cap, wherein the design of the material thickness and shape of the cap can alter the auditory impression of the acoustic signal within certain limits In alternative embodiments, the sealing cap may not form a resonating body.

In some embodiments, the signal member has a first engagement member, which is in particular resilient or/and is arranged on a resilient arm, which member releasably engages in the drive element, in particular in a recess of the drive element. As a result, the drive element is coupled to the signal member in an axially fixed manner, wherein the axially fixed coupling between the drive element and the signal member is released when the signal member, in particular the first engagement member, is disengaged or pressed out of the engagement with the drive element, in particular the recess of the drive element. In particular, the first engagement element is released from engagement with the propulsion element at the end of the first partial stroke of the propulsion element.

The signal stop is arranged along the longitudinal axis of the autoinjector such that it is arranged in alignment with the signal member. This causes the signal member to hit the signal stop with a movement along the longitudinal axis of the autoinjector.

In embodiments with a needle protection sleeve, it may be that the needle protection sleeve acts on the second spring, wherein the needle protection sleeve is displaceable from its initial position relative to the housing and along the longitudinal axis of the autoinjector in the proximal direction, i.e. against the direction of dispensing, in particular by the actuating stroke, for triggering the product dispensing. As a result, the second spring is tensioned or biased, and optionally also the discharge of the product, in particular the movement of the drive element in the discharge direction, is triggered. The needle shielding sleeve may be moved from its initial position by the actuating stroke into its actuated position in that its distal end is pressed against the puncture site of the patient, wherein the housing is moved relative to the needle shielding sleeve in the direction of the puncture site, so that the needle shielding sleeve executes the actuating stroke relative to the housing. The needle protruding from the distal end of the needle protection sleeve is also inserted into the puncture site. After the discharge of the product has been carried out, in particular after a short waiting time, for example 3 to 10 seconds, after which the signal has been generated by means of the signal member, the autoinjector is removed from the puncture site, whereby the needle shielding sleeve is moved by the needle shielding stroke relative to the housing from its actuated position into the needle shielding position, in particular by means of the spring energy stored in the second spring. Removing the autoinjector from the injection site also removes the needle from the injection site.

In certain embodiments, a switching module can be arranged kinematically between the second spring and the needle shielding sleeve, wherein the switching module is entrained by the needle shielding sleeve in the proximal direction when the needle shielding sleeve is moved from its initial position in the proximal direction or into the actuated position, and moves the needle shielding sleeve in the distal direction when the spring acting on the switching module moves the switching module in the distal direction. The switching module or a part thereof, for example a switching sleeve, can be integrally connected with the needle shielding sleeve or, for example, connected in a positive-locking manner, for example snap-fit, or loosely resting against the needle shielding sleeve. The switching module can be a single part or can comprise a plurality of parts, wherein a multi-part switching module can comprise at least the switching sleeve and a locking sleeve. The locking sleeve can be movable relative to the needle shielding sleeve and/or switching sleeve, for example along the longitudinal axis. For example, the second spring can brace against the switching sleeve and the switching sleeve against the needle shielding sleeve. In alternative embodiments, the switching sleeve and needle protection sleeve can be connected in an axially fixed and optionally rotationally fixed manner, wherein the second spring can be supported on the switching sleeve. In further alternative embodiments, the switching sleeve and needle protection sleeve can be designed in one piece, wherein the second spring can be supported on the switching sleeve. Between the locking sleeve and the switching sleeve, a, for example, unidirectionally acting locking member can be provided which optionally the aforementioned locking member that locks the needle shielding sleeve in its needle shielding position and is formed, for example, by the locking sleeve and engages in the switching sleeve, for instance in a recess. The locking member may be configured such that the switching sleeve during its movement relative to the housing in the proximal direction, entrains the locking sleeve via the locking member, for instance during the movement of the needle shielding sleeve out of its initial position into the actuated position and, during its movement relative to the housing in the distal direction relative to the locking sleeve, is moved into a locking position, for instance during the movement of the needle shielding sleeve from its actuated position into the needle shielding position, wherein in the locking position the locking member or another locking member, such as the further aforementioned one, blocks a movement of the switching sleeve relative to the locking sleeve in the proximal direction. This advantageously prevents the needle shielding sleeve from being pushed back into the housing from its needle shielding position for renewed release of the needle tip.

For example, the switching sleeve can have a first recess in which the locking member of the locking sleeve engages releasably when the needle shielding sleeve is moved out of its initial position into its actuated position. For example, the switching sleeve can have a second recess in which the locking member or possibly the other locking member engages when the needle shielding sleeve is in its needle shielding position. The first and second recess can be arranged at a distance, which approximately corresponds to the needle shielding stroke, along the longitudinal axis. Of course, a reversal of the arrangement of the recesses and of the locking member or of the locking members is also possible, i.e., the at least one locking member can be formed on the switching sleeve and the at least one recess, i.e., the first recess and, where applicable, the second recess on the locking sleeve.

In alternative embodiments, the switching sleeve can have a first recess in which the locking member or locking members of the locking sleeve engage(s) releasably when the needle shielding sleeve is moved from its initial position into its actuated position. The locking member of the locking sleeve can hit the proximal end of the switching sleeve when the needle shielding sleeve is in its needle shielding position. Of course, a reversal of the arrangement of the recess and the locking member is also possible.

The locking member and, where applicable, the other locking member can be arranged resiliently, for instance in each case on a resilient arm. The switching sleeve can surround and/or guide the locking sleeve.

In some embodiments, the signal member can have a second engagement member, which can be moved into an, for instance axially fixed, engagement with the needle shielding sleeve or the switching module, for instance the locking sleeve, by the disengagement movement of the first engagement member with which the first engagement member moves out of the drive element. The first engagement member and the second engagement member of the signal member can be matched to one another in such a way that the second engagement member already engages, optionally in an axially fixed manner, in the needle shielding sleeve or the switching module when the first engagement member is not yet completely released from the engagement with the drive element. This advantageously reliably prevents the first engagement member from being released from the engagement with the drive element already when the second engagement member is not yet in the engagement with the needle shielding sleeve or the switching module, for instance the locking sleeve. The needle shielding sleeve or the switching module, for instance the locking sleeve, can have a further recess, for example, into which the second engagement member of the signal member engages, for example for the axially fixed coupling between the signal member and the switching module, for instance the locking sleeve or the needle shielding sleeve. The drive element can have a recess in which the first engagement member engages for the axially fixed coupling between the drive element and the signal member. The first engagement member and the second engagement member are formed on a common elastic arm, wherein the first engagement member points, for example, radially toward the longitudinal axis and the second engagement member points, for example, radially away from the longitudinal axis. The first and second engagement members can be arranged, optionally radially, between the drive element and the needle shielding sleeve or the switching module, for instance the locking sleeve.

For instance, during the discharge stroke of the drive element, for instance at the end of the first partial stroke, the first engagement member of the signal member is released from the engagement with the drive element, and, at the same time, the second engagement member of the signal member is brought into engagement with the switching module or the needle shielding sleeve, for instance with a movement transverse to the longitudinal axis. For instance, the drive element, by virtue of its movement in the discharge direction, can press the first engagement member out of the recess of the drive element and the second engagement member into the recess of the needle shielding sleeve or the switching module, for instance the locking sleeve.

In some embodiments, the needle shielding sleeve or the switching module, for instance the locking sleeve, can hold the first engagement member of the signal member in the engagement with the recess of the drive element, wherein the recess for the second engagement member of the signal member is moved toward the second engagement member by moving the needle shielding sleeve from its initial position into its actuated position, wherein the recess in the actuated position of the needle shielding sleeve, for instance in the instant at which the discharge stroke is enabled, is arranged at a distance along the longitudinal axis, which corresponds approximately to the first partial stroke of the signal member, from the second engagement member. The propulsion element released for the discharge stroke by actuating the needle protection sleeve can then be moved by the first partial stroke in the direction of discharge. The first engagement member is held in the engagement with the drive element by the inner circumference of the needle shielding sleeve or of the switching module, for instance of the locking sleeve, against which the second engagement member rests. The second engagement member is located at the end of the first partial stroke relative to the longitudinal axis at the same position as the recess, whereby the second engagement member can engage in its recess and move the first engagement member out of its recess.

The discharge stroke of the drive element can comprise for instance two phases, namely the first partial stroke and the second partial stroke. During the first partial stroke, the first engagement member of the signal member is in the axially fixed engagement with the drive element and the second engagement member of the signal member is out of the axially fixed engagement with the needle shielding sleeve or the switching module, for instance the locking sleeve. During the second partial stroke of the discharge stroke, the second engagement member is in the axially fixed engagement with the needle shielding sleeve or the switching module, for instance the locking sleeve, wherein the first engagement member is out of the engagement with the drive element, whereby it is advantageously brought about that the drive element is movable relative to the signal member in the distal direction by means of the first spring and/or the signal member is not yet released for the signal triggering.

In alternative embodiments, the discharge stroke can comprise three phases, for instance a further partial stroke. During the first partial stroke, the drive element moves axially relative to the signal member. During the first partial stroke, the signal member is not charged. During the second partial stroke, the first engagement member of the signal member is in the axially fixed engagement with the drive element and the second engagement member of the signal member is out of the axially fixed engagement with the needle shielding sleeve or the switching module, for instance the locking sleeve. During the second partial stroke, the signal member is charged. During the third partial stroke of the discharge stroke, the second engagement member is in the axially fixed engagement with the needle shielding sleeve or the switching module, for instance the locking sleeve, wherein the first engagement member is out of the engagement with the drive element, whereby it is advantageously brought about that the drive element is movable relative to the signal member in the distal direction by means of the first spring and/or the signal member is not yet enabled for the signal triggering.

The drive element may be movable relative to the signal member in the distal direction by means of the first spring, for instance by the second partial stroke, when the first engagement member of the signal member is out of the engagement with the drive element and the second engagement member of the signal member is in the engagement with the needle shielding sleeve or the switching module.

In some embodiments, the second engagement member of the signal member and the recess for the second engagement member can be arranged in the delivery state of the autoinjector along the longitudinal axis approximately at the distance from each other which is approximately the sum of the actuating stroke of the needle shielding sleeve and the first partial stroke of the drive element, which approximately corresponds to the stroke of the signal member away from the signal stop.

The drive element can prevent the second engagement member of the signal member from moving out of the axially fixed engagement with the needle shielding sleeve or the switching module when the drive element moves relative to the signal member in the distal direction, for instance during the second partial stroke of the drive element. The drive element allows the second engagement member at the end of the discharge stroke or the second partial stroke to move out of the engagement with the needle shielding sleeve or the switching module. If the second engagement member is moved out of the engagement with the needle shielding sleeve or the switching module at the end of the second partial stroke, the signal member is accelerated by the second spring counter to the discharge direction and hits against the signal stop. The second engagement member is held in the engagement with the needle shielding sleeve or the switching module by the outer circumference of the drive element, against which the first engagement member rests.

In some embodiments, the autoinjector can have a holding element against which, for example, one end of the first spring, for instance the proximal end of the first spring, braces. Alternatively, the proximal end of the spring can brace against the housing or an element fixed to the housing. The holding element itself can be arranged fixed to the housing or movably in relation to the housing. The holding element can have a first engagement element which, before the triggering of the discharge of the product, engages in the drive element, whereby the drive element is prevented from moving relative to the holding element and/or the housing in the discharge direction. The engagement of the first engagement element in the drive element is releasable for the discharge of the product. When the engagement is released, the drive element is enabled for the movement in the discharge direction. The first spring can move the drive element relative to the holding element and/or the housing by the discharge stroke in the discharge direction. The drive element can have a recess for the first engagement element of the holding element, wherein this coupling between the drive element and the holding element is released when the holding element, for instance the first engagement element, is moved out of the engagement with the drive element, for instance the recess of the drive element. For instance, the first engagement element can thereby be released from the engagement with the drive element in that the needle shielding sleeve is moved from the initial position by the actuating stroke into the actuated position. For example, the first engagement element can be held by the needle shielding sleeve or the switching module, for instance the locking sleeve, in the axially fixed engagement with the drive element when the needle shielding sleeve is not in its actuated position or in its initial position. For example, an inner circumference of the needle shielding sleeve or of the switching module, for instance of the locking sleeve, can hold the first engagement element in the engagement with the drive element, wherein, for example, a second engagement element, which is described further below, can rest against the inner circumference.

By moving the needle shielding sleeve into its actuated position, the needle shielding sleeve or the switching module, for instance the locking sleeve, can allow the first engagement element of the holding element to be moved out of the engagement with the drive element, for instance with a movement transverse to the longitudinal axis of the autoinjector. For example, a recess, for instance for the second engagement element of the holding element, which is formed on the needle shielding sleeve or the switching module, for instance of the locking sleeve, can be arranged in the same position in relation to the longitudinal axis, like the first and/or second engagement element, so that the first engagement element can move out of the engagement with the drive element. For example, the drive element can press the first engagement element out of the engagement with the drive element when the needle shielding sleeve is in its actuated position.

The first engagement element of the holding element can, for example, point radially to the longitudinal axis and/or be arranged on a resilient arm of the holding element.

As mentioned, the holding element can have a second engagement element, which can be moved out of the propulsion element into axially fixed engagement with the needle protection sleeve or the switching module, such as the locking sleeve, by the disengagement movement of the first engagement element. The second engagement element can, e.g., be arranged on the arm on which the first engagement element is arranged and/or, e. g., can point radially away from the longitudinal axis. The first engagement element and the second engagement element can be matched to one another in such a way that the second engagement element already engages axially fixedly in its recess, which is formed by the needle shielding sleeve or the switching module, for instance the locking sleeve, when the first engagement element is not yet completely released from the engagement with the drive element. As a result, it is advantageously achieved that first the axially fixed connection between the holding element and the needle shielding sleeve or the switching module is produced before the axially fixed connection between the holding element and the drive element is released and thus a renewed or further pushing back of the needle shielding sleeve is blocked.

For instance when the second engagement element of the retaining element is in its recess, the drive element can move in the distal direction relative to the holding element, for instance due to the energy stored in the biased spring. The drive element can prevent the second engagement element from moving out of the axially fixed engagement with the needle shielding sleeve or the switching module, for instance the locking sleeve, when the drive element moves relative to the signal member in the distal direction. This optionally also applies at the end of the discharge stroke, for instance also when the second engagement member of the signal member is released from its recess in order to be accelerated by the second spring counter to the discharge direction.

For instance in embodiments in which the recess for the second engagement element of the holding element is formed by the needle shielding sleeve or the switching sleeve, it is optionally for the second engagement element to move out of its recess at the end of the discharge stroke in order to be able to move the needle shielding sleeve from the actuated position into the needle shielding position after the product has been administered. For this purpose, the drive element can have a recess into which the first engagement element can engage, wherein the second engagement element simultaneously moves out of its recess in order to enable the movement of the needle shielding sleeve in the distal direction.

In embodiments with a switching module having a switching sleeve and a locking sleeve, it is optionally that the second engagement element also remains at the end of the discharge stroke such that the second engagement element prevents the locking sleeve from being moved in the distal direction relative to the housing and/or the second engagement element, wherein the switching sleeve and/or the needle protection sleeve are displaceable in the distal direction relative to the locking sleeve, for instance due to the energy stored in the second spring, whereby the needle protection sleeve is moved into its needle protection position. As already described and only noted for the sake of completeness, the locking member can enter into engagement between the locking sleeve and the switching sleeve, which prevents the switching sleeve from being movable relative to the locking sleeve in the proximal direction. A movement of the locking sleeve in the proximal direction is prevented by the locking sleeve hitting either against the housing or against an element fixed to the housing, such as, for example, on a mechanism holder or the signal member.

A further aspect of the invention relates to the embodiment of a product container holder, for instance a tip holder for an autoinjector, for instance for an autoinjector in which the product container is non-displaceable with respect to the housing or for an autoinjector of the type described above.

The invention is based on a syringe module which is intended for use in an autoinjector. For instance, an autoinjector can be provided which has such a syringe module. The syringe module comprises a syringe and a syringe holder. The syringe has a syringe body, a plunger and a needle, wherein the needle is fastened, for example, non-detachably to a needle holding portion of the syringe and the plunger is arranged displaceably in a cylindrical portion of the syringe body, wherein the syringe body has a tapered portion or area which is arranged between the needle holding portion and the cylindrical portion. The syringe further has a needle protection cap, which can be a so-called soft needle shield or alternatively a rigid needle shield, for example. A soft needle shield may be formed from a rubber-elastic plastic, wherein a rigid needle shield is formed from a sleeve made of hard plastic, in which a sleeve made of a rubber-elastic plastic is arranged. The sleeve made of rubber-elastic plastic and the sleeve made of hard plastic together form the rigid needle shield. The needle protection cap, which covers the needle and is fastened to the needle holding section, which extends conically towards the needle tip, and may keep the needle protected from dirt and sterile. A gap is formed between the tapering portion and the needle shielding cap, such as the sleeve made of hard plastic.

The syringe holder has at least one engagement element, such as a shoulder, on which the tapered portion of the syringe is supported in the distal direction and which engages in the gap between the needle protection cap and the tapered portion. Advantageously, the contact of the tapered portion with the at least one shoulder prevents the syringe from moving in the distal direction relative to the syringe holder.

There may be a gap between the shoulder and the needle protection cap so that the needle protection cap remains unloaded by the shoulder. This advantageously prevents the sterility of the needle from being impaired by unintentional displacement of the needle protection cap by the shoulder.

In some embodiments, the syringe body may have a finger flange at its proximal end, wherein a gap is formed between the finger flange and the syringe body when the tapered portion is in contact with the shoulder, leaving the finger flange substantially unloaded. This advantageously prevents the finger flange from being overloaded and breaking the syringe body.

The syringe holder may have at least one retaining member, for instance an outwardly directed projection, with which the syringe holder can be connected or is connected in an axially fixed manner to a housing of the autoinjector, for instance can be snapped or snapped together.

For instance, the syringe holder may have at least one cam which is resiliently arranged on an arm, and is arranged, for example, distally of the holding member. The at least one cam can inhibit or prevent a needle protection sleeve from moving from its starting position into its actuated position in such a way that when a limit force exerted on the needle protection sleeve along the longitudinal axis L of the autoinjector is exceeded, the at least one cam is pressed out of engagement with the needle protection sleeve, as a result of which the needle protection sleeve can be displaced abruptly into its actuated position relative to the housing.

The housing of the autoinjector may, for example, have a retaining section which bears against the syringe holder, such as against an outer surface or an outer circumference of the syringe holder, and prevents the at least one engagement element of the syringe holder from moving away from the longitudinal axis transversely to the longitudinal axis. For instance, the retaining portion may be cylindrical and surround the at least one engagement element, such as two or three or four engagement elements, so that the at least one engagement element is arranged within the holding portion. For mounting or inserter of the syringe in the syringe holder, the syringe holder is outside the engagement with the holding section of the housing. When the syringe is fully inserted into the syringe holder, for instance when the at least one engagement element engages in the gap between the tapered portion and the needle protection cap, the syringe module or the syringe holder is brought into engagement with the holding portion so that the at least one engagement element is prevented from moving out of engagement with the tapered portion transversely to the longitudinal axis, such as away from the longitudinal axis or outwards.

In a first variant, the at least one engagement element can be formed resiliently on an arm on the syringe holder, wherein the syringe is inserted into the syringe holder, which may be sleeve-shaped, via the proximal end with the needle in front, wherein the needle protection cap deflects the at least one engagement element transversely to the longitudinal axis outwards, i.e. away from the longitudinal axis, wherein, when the needle protection cap has been moved completely past the at least one engagement element, the at least one engagement element snaps into the gap between the tapered area and the needle protection cap. Subsequently, the syringe holder with the syringe is displaced into engagement with the retaining portion of the housing of the autoinjector, whereby the at least one engagement element is held in engagement with the gap between the needle protection cap and the tapered portion and can no longer spring out of this engagement.

The housing comprises a holding portion. The holding portion of the housing can be designed in the shape of a sleeve and arranged inside the housing. A stop for limiting the axial movement of the needle protection sleeve in the proximal direction can be provided between the sleeve-shaped holding portion of the housing and the sleeve-shaped housing. The holding portion of the housing projects beyond the distal end of the housing in the distal direction. Further, the holding portion may have one or more grooves which, in connection with the rail or rails provided on the needle protection sleeve, form an anti-rotation lock between the housing and the needle protection sleeve. The rail of the needle shielding sleeve may be provided on a sleeve inner surface of the needle shielding sleeve. The groove of the holding portion may be arranged on a sleeve outer surface of the holding portion. The needle shielding sleeve can be axially moved in a rotationally fixed manner between the holding portion of the housing and the housing. In alternative embodiments, the one or more grooves can be provided on the needle shielding sleeve and the one or more rails can be provided on the holding portion of the housing in order to form a locking connection between the housing and the needle shielding sleeve. The groove of the needle shielding sleeve may be provided on a sleeve inner surface of the needle shielding sleeve. The rail of the holding portion may be arranged on a sleeve outer surface of the holding portion.

The autoinjector according to the invention has an electronic module with a sensor. The sensor is configured to measure the axial movement of the signal element from a position at the start of the discharge, wherein the signal element is entrained in the direction of discharge, to a position at the end of the discharge, wherein the signal element strikes the signal stop. The electronic module is installed in the autoinjector by the manufacturer at the time of delivery to the user or patient. The user does not have to install the electronic module himself and cannot use it more than once.

By providing the signal element, which can be moved axially relative to the housing, the injection can be carried out directly by monitoring the positions, such as the position at the start of the distribution and the position at the end of the distribution of the signal element. Two position changes can be recorded or detected.

This monitoring of the signal element's positions via the electronic module can be considered as a supplement or as an alternative to the aforementioned acoustic and/or tactile signal generation in order to improve the patient's therapy.

In alternative embodiments, the monitoring of the positions of the signal member can be performed only via the electronic module, wherein the aforementioned acoustic and/or tactile signal generations are not realized in the auto-injector or are actively suppressed or at least attenuated. A feedback signal to the user is generated electronically in its location.

In some embodiments, the sensor of the electronic module comprises a switch or a button that senses or detects the positional displacement of the signal element. The sensor can be designed as a switching detector. The autoinjector can comprise a switch actuator to actuate the switch or the switch detector button. The switching detector can be fastened to the electronic module in a fixed manner, such as axially and rotationally. The switching actuator can be designed as a separate element or alternatively be provided on the signal element or alternatively the signal element can be designed as a switching actuator or alternatively be provided on the cap of the autoinjector, on the housing of the autoinjector, on the electronic module or on another housing of the electronic module or alternatively the cap of the autoinjector, the housing of the autoinjector, the electronic module or the other housing of the electronic module can be designed as a switching actuator.

In one embodiment, the switching actuator can be movable, such as axially movable, especially axially movable and rotationally fixed in the autoinjector. The switching actuator can be arranged so as to be axially movable, displaceable or slidable relative to the housing of the autoinjector, the sealing cap of the autoinjector, the electronic module or the other housing of the electronic module. The switching actuator can be axially movable, displaceable or slidable in the housing of the autoinjector, the cap of the autoinjector, the electronic module or the other housing of the electronic module. The switching actuator may be a separate element, such as a pin-shaped element, wherein the switching actuator can be entrained by the signal element in the proximal direction during movement of the signal element, whereas the switching actuator can move in the distal direction during movement of the signal element in the distal direction due to the force of gravity, which is greater than the frictional force between the switching actuator and the housing of the autoinjector, the cap of the autoinjector, the electronic module or the other housing of the electronic module. If the signal element is in a proximal position, such as a start and/or end position, the switching actuator is also in a proximal position. If the signal element is in a distal position, the switching actuator is also in a distal position. The axial movement of the signal element in the distal direction at the start of the distribution and the axial movement of the signal element in the proximal direction at the end of the distribution can thus be transmitted from the switching actuator to the switching detector.

The switching detector is configured to measure the axial movement of the switching actuator from a position at the start of the discharge, wherein the switching actuator is movable, displaceable or slidable in the direction of discharge, to a position at the end of the discharge. The switching detector can thus detect the position of the signal element.

Alternatively or additionally, the switching detector can have a preload force, such as a spring force, or be designed to be preloaded, for instance resilient. Alternatively or cumulatively to the force of gravity, this force can thus cause the switching actuator to be moved or to move in the distal direction during the movement of the signal element in the distal direction. The preload force may be greater than the gravitational force of the switching actuator so that no gravity-driven proximal movement of the switching actuator leads to premature detection of the start of the pouring movement when injecting against gravity.

If, following injection, the electronic module is detached from the autoinjector by the user or a disposal specialist, this detachment movement can also be detected by the switching detector, provided that the switching actuator itself does not remain in its proximal position relative to the switching detector on the electronic module. Such a bayonet-like separating movement can comprise a movement of the switching detector radially or tangentially away from the axis or the switching actuator.

In another embodiment, the switching actuator can be designed in the shape of a lever. A pivot point of the lever-shaped switching actuator or a pivot joint of the lever-shaped switching actuator can be at least partially accommodated in the housing of the autoinjector, in the cap of the autoinjector, in the electronic module or in the other housing of the electronic module. The switching actuator is axially fixed and rotatable relative to the housing of the autoinjector, the sealing cap of the autoinjector, the electronic module or the other housing of the electronic module. The other part of the lever-shaped switching actuator is arranged between the signal element and the switching detector in such a manner that at least the axial movement of the signal element in the distal direction at the start of the distribution and the axial movement of the signal element in the proximal direction at the end of the distribution can be transmitted to the switching detector via the movement of the switching actuator. The switching actuator can redirect or divert a movement of the signal element to be detected.

In another embodiment, the switching actuator, for instance the lever-shaped switching actuator, can be at least partially biased, for instance designed to be elastically biased or resilient. The switching actuator can be accommodated at least partially in the housing of the autoinjector, in the cap of the autoinjector, in the electronic module or in the other housing of the electronic module. The switching actuator is arranged or fastened axially and rotationally fixed relative to the housing of the autoinjector, the sealing cap of the autoinjector, the electronic module or the other housing of the electronic module. The other part of the pretensionable, such as the elastically pretensionable or the resilient switching actuator is arranged between the signal element and the switching detector in such a way that the axial movement of the signal element in the distal direction at the start of the distribution and the axial movement of the signal element in the proximal direction at the end of the distribution can be transmitted to the switching detector via the movement of the switching actuator. The switching actuator can be preloaded, such as elastically preloaded, between the switching detector and the signal element before the start of the distribution and at the end of the distribution, wherein the stored energy of the switching actuator is released during the movement of the signal element in the distal direction such that the switching actuator relaxes and/or is moved in the distal direction during the start of the distribution. Thus, the switching actuator, for instance the preloadable, especially the elastically preloadable or the resilient switching actuator, can suitably forward or transmit a movement of the signal element to be detected.

In another embodiment, the switching actuator and the housing of the autoinjector, the cap of the autoinjector or the other housing of the electronic module can be designed in one piece. Part of the housing of the autoinjector, the sealing cap of the autoinjector or the other housing of the electronic module can be designed to be pretensionable, such as elastically pretensionable or resilient, or have a pretensionable arm, such as a spring arm. The switching actuator is arranged in the tensionable, for instance in the elastically pretensionable or in the resilient part of the housing of the autoinjector, the sealing cap of the autoinjector or the other housing of the electronic module in such a way that it can suitably deflect or redirect the movement of the signal element to be detected to the switching detector. The pretensionable, such as the elastically pretensionable or the resilient part of the housing of the autoinjector, the sealing cap of the autoinjector or the other housing of the electronic module can be pretensioned, such as elastically pretensioned, between the switching detector and the signal element before the start of the distribution and at the end of the distribution. The stored energy of the pre-stressed, such as the elastically pre-stressed part of the housing of the autoinjector, the cap of the autoinjector or the other housing of the electronic module can be released during the movement of the signal element in the distal direction in such a way that the switching actuator relaxes and/or moves in the distal direction during the start of the distribution. The switching actuator can thus suitably forward or pass on a movement of the signal element to be detected to the switching detector.

In other embodiments, the sensor is designed as an optical sensor, force sensor, magnetic sensor, inductive sensor or electrically conductive sensor. This depends on the subject matter that is to be detected.

In another embodiment, an electrically conductive switching actuator can be provided. The switching actuator can be designed to be fully or only partially electrically conductive. The electrically conductive part of the switching actuator can, for example, be applied by means of a coating process or produced as a two-component part. The electrically conductive switching actuator can be axially connected to the signal element, designed in one piece with the signal element or the switching actuator and the signal element can be separate parts, wherein the switching actuator and the signal element can be moved or displaced axially at the same time or the movement of the signal element is transmitted to the switching actuator. The electronic module can have at least two electrical contact points, wherein an electrical circuit can be closed when the electrically conductive switching actuator is in contact with the two electrical contact points and the electrical circuit can be open when there is a distance between the electrically conductive switching actuator and the two electrical contact points. The electronic module can thus suitably detect or detect the position of the signal element.

In a further embodiment, the electrically conductive switching actuator can be designed as an electrically conductive contact plate or have an electrically conductive contact plate. The electronic module can have at least one electrical contact. One end of the electrical contact plate can always be connected to the at least one electrical contact of the electronic module, wherein the other end of the electrical contact plate can form a closed or an open electrical circuit depending on the axial position of the switching actuator or the signal element. For example, the electrical circuit can be closed when the signal element or/and the switching actuator are in the proximal position, whereas the electrical circuit is open when the signal element or/and the switching actuator are in the distal position. The electrical contact plate can be pretensionable, such as elastically pretensionable or resiliently designed.

A common feature of all embodiments is that the autoinjector has an electronic module with a suitable sensor for measuring the axial movement of the signal element from a position at the start of the distribution to a position at the end of the distribution. The electronic module can therefore be used to detect the start and/or end of injection.

The electronic module can be designed as a separate electronic module and can be detachably connected to the housing and/or sealing cap of the autoinjector, for instance by means of a bayonet lock to the housing and/or sealing cap of the autoinjector. Other detachable connections, such as snap locks or twist locks, can also be provided between the electronic module and the housing and/or cap of the autoinjector. Advantageously, the electronic module can be designed as reusable and the autoinjector as single-use. In addition, the electronic module and the autoinjector can be disposed of separately to ensure environmentally friendly disposal. For this purpose, a separating washer can be provided between the electronic module and the housing and/or sealing cap. The separating disk is used to safely separate and/or prevent premature separation of the electronic module from the housing and/or sealing cap.

In alternative embodiments, the electronic module is firmly, for instance non-detachably, connected to the housing and/or sealing cap of the autoinjector. The advantage is that operating errors by the patient are avoided, thus ensuring safe use of the autoinjector.

The electronic module can be accommodated by an electronic module housing. The above-mentioned other housing of the electronic module can be designed as an electronic module housing. The electronic module housing is used to protect the electronic module. In addition, the electronic module housing can be designed in such a way that the electronic module can be easily and safely removed from the housing and/or sealing cap. For this purpose, a bayonet lock or another detachable connection, such as a snap lock or twist lock, can be provided between the electronic module housing and the housing and/or sealing cap. The separating disk is used to safely separate and/or prevent premature separation of the electronic module housing from the housing and/or sealing cap.

The separating disk can be mounted in the housing and/or the sealing cap in such a way that the electronic module and/or the electronic module housing can only be removed from the housing and/or the sealing cap after triggering and/or at the end of the discharge.

In some embodiments, the electronic module comprises an energy source and a processor, wherein the processor is coupled to the sensor and to the energy source and configured such that the start and/or the end of the discharge of the autoinjector can be registered or sensed by means of the sensor. The axial movement of the signal element from a position at the start of the distribution to a position at the end of the distribution is recorded or detected.

The energy source can be designed as a battery. It can be a disposable battery or a rechargeable battery or accumulator.

The electronic module can comprise a switch for activating the electronic module. This means that the electronic module must be switched off before the injection and only switched on during the injection. This saves energy from the energy source.

The processor may have evaluation electronics configured to identify processes in or states of the injection device during an injection process based on measurements from the sensor. For example, axial movements can be filtered and compared with a threshold value in a comparator or a comparator circuit. If a pattern determined in this manner matches a predetermined pattern, the axial movement is identified as belonging to the determined event.

The electronic module can comprise a timer that measures the duration of the autoinjector's discharge.

The processor of the electronic module can be configured to operate the timepiece. The processor of the electronic module may be configured to register or sense the elapsed time since the last distribution and/or to register or sense after a fixed duration after the registered or sensed end of the distribution and/or to register or sense after the registered start of the distribution.

In some embodiments, the electronic module may contain a wireless communication unit for communicating with an external electronic apparatus and/or a status indicator for indicating at least one position of the signal element.

In some embodiments, the status indicator can be activated after a fixed duration after the registered or sensed end and/or after the registered or sensed start of the distribution.

The status indicator can be designed as an electrical light source, for example as an LED. However, another visual signal generator and/or an acoustic signal generator for generating sounds or melodies and/or a tactile signal generator for generating movements can also be provided. The status indicator can display information on the state of the autoinjector. The start and/or end of the distribution can be displayed. Further, the expiry of a holding time or a waiting time can be displayed additionally or alternatively, wherein the patient can remove the autoinjector from the skin after this time has elapsed.

The electronic module housing may comprise a light source housing, for example an LED housing, to better indicate the state to the patient. Alternatively, the electronic module housing can form the light source housing, for example the LED housing. The light source housing can be designed as a light guide.

Alternatively and/or additionally, the time recording function and/or the status indicator can be performed by real-time event transmission to the external electronic apparatus.

The external electronic apparatus can be a computer, a cloud and/or a mobile device comprising a smartphone or a smartwatch.

In alternative embodiments, the autoinjector may have an additional or alternative electronic module with an additional or alternative sensor configured to measure the axial movement of the needle protection sleeve and/or propulsion element and/or switching sleeve and/or locking sleeve. This electronic module can be designed as a separate electronic unit or as an electronic unit integrated into the autoinjector.

There can be at least two autoinjectors, wherein the corresponding electronic modules are different in order to measure a desired function of the autoinjector. For instance, a first and a second autoinjector may be provided, wherein the first electronic module of the first autoinjector is different from the second electronic module of the second autoinjector, and that the autoinjectors are otherwise identical.

### BRIEF DESCRIPTION OF THE DRAWINGS

The invention and the further aspect with respect to the invention have been described with reference to several embodiments. In the following, some embodiments are described with reference to figures. The features disclosed herein advantageously further form the subject matter of the invention individually and in any combination of features. In the drawings:

FIG. 1 shows an exploded view of an autoinjector in accordance with an embodiment,

FIGS. 2a-2c show the autoinjector of FIG. 1 in a delivery state, wherein FIGS. 2a to 2c are sectional views extending through the longitudinal axis of the device, the sectional views being angularly offset about the longitudinal axis,

FIGS. 3a-3c show the device and the views from FIGS. 2a-2c, wherein a needle protection sleeve is in its actuated position, generating a signal signaling the start of product dispensing.

FIGS. 4a-4c show the device and the views from FIGS. 2a-2c, wherein a signal signaling the end of product dispensing is generated,

FIGS. 5a-5c show the device and the views from FIGS. 2a-2c, wherein the needle protection sleeve is in its needle protection position,

FIGS. 6a-6c shows a side, top and perspective view, respectively, of the electronic module (16) from FIG. 1.

FIGS. 7a-7c show longitudinal sectional views of an alternative embodiment of an autoinjector according to the invention.

### DETAILED DESCRIPTION

The term “drug” as used herein comprises any flowable medical formulation suitable for controlled administration through a means such as a cannula or hollow needle, for example comprising a liquid, a solution, a gel, or a fine suspension containing one or more active medical ingredients. A medication can be a single active ingredient composition or a pre-mixed or co-formulated composition having a plurality of active ingredients from a single container. Medication comprises drugs such as peptides (e.g., insulins, insulin-containing medications, GLP-1-containing and derived or analogous preparations), proteins and hormones, biologically obtained or active ingredients, active ingredients based on hormones or genes, nutrient formulations, enzymes and further substances both in solid (suspended) or liquid form but also polysaccharides, vaccines, DNS or RNS or oligonucleotides, antibodies or parts of antibodies, and suitable base, auxiliary and excipient substances.

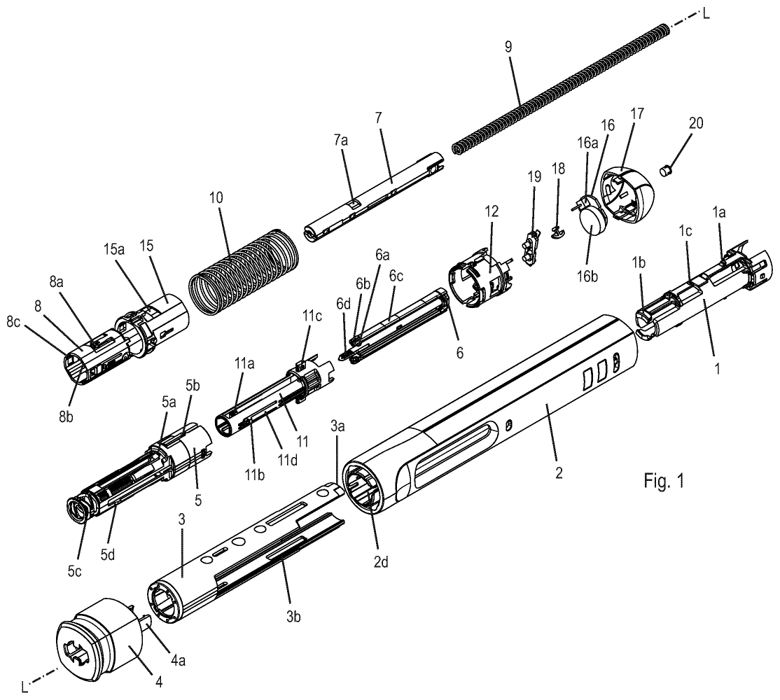

With reference to FIGS. 1-7c, the structural features and function of some autoinjectors are now described.

In one embodiment of the invention, which is represented in FIGS. 1-6, the autoinjector has a sleeve-shaped, elongated housing 2 with a longitudinal axis L, which has a closure cap 12 at its proximal end, which is connected to the housing 2 in a rotationally and axially fixed manner The sealing cap 12 is firmly connected to the housing 2, for example via a latching connection.

A detachable electronic module housing 17 can be provided at the proximal end of the sealing cap 12.

A bayonet lock may be provided between the housing 2 and/or the sealing cap 12 and the electronic module housing 17 in order to remove the electronic module housing 17 from the autoinjector. Other detachable connections, for example a snap lock or a twist lock, can also be provided between the electronic module housing 17 and the housing and/or sealing cap of the autoinjector.

In alternative embodiments, the electronic module housing 17 may be fixedly connected to the housing 2 and/or the cap 12, wherein the electronic module housing 17 is not removable from the housing 2 and/or the cap 12 of the autoinjector.

An electronic module 16 is provided in the electronic module housing 17. Thus, the electronic module 16 can be detachably or non-detachably connected to the housing 2 and/or the sealing cap 12.

Alternatively, the electronic module can be detachably or non-detachably arranged at a different location in the autoinjector. The arrangement depends on the subject matter to be detected by the autoinjector.

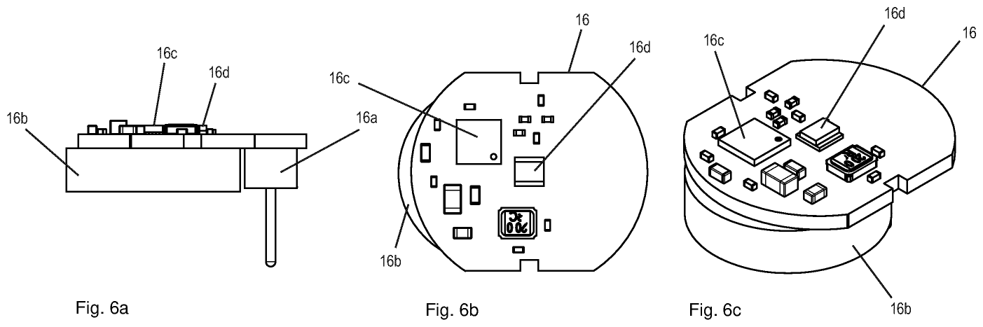

The electronic module 16 comprises a sensor 16a, a battery 16b, a processor 16c and a light source 16d (FIGS. 6a, 6b, 6c). The light source 16d can be designed as an LED.

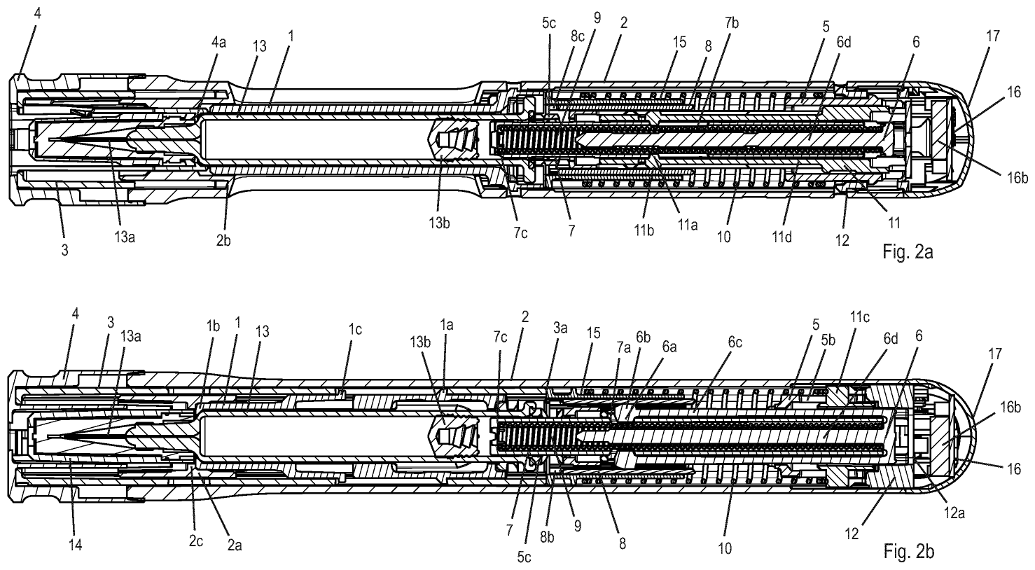

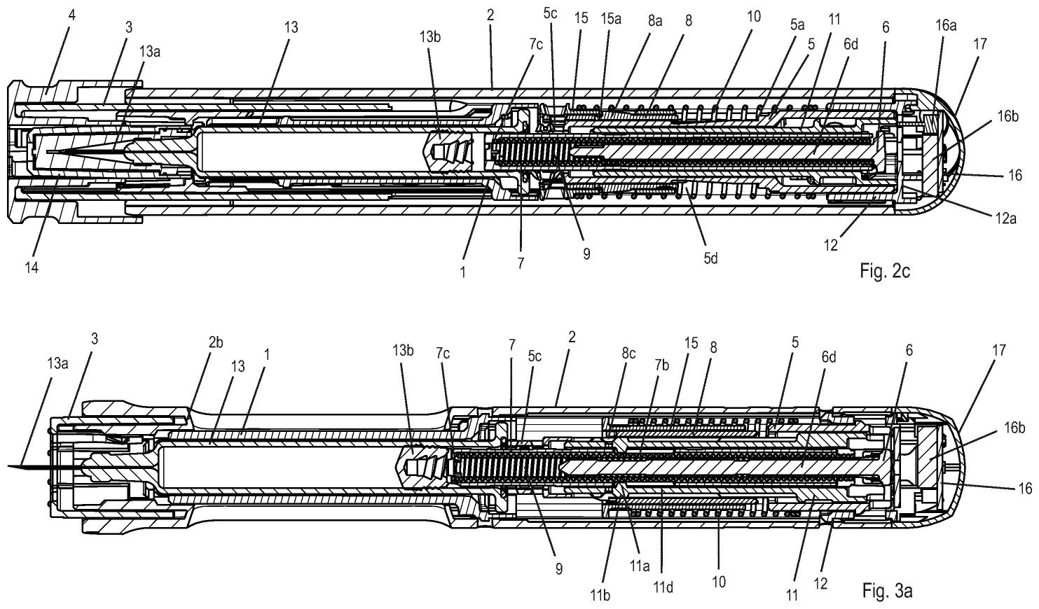

In its as-delivered state (FIGS. 2a-2c), a pull-off cap 4 is arranged at the distal end of the autoinjector, which is pulled off, or twisted off, and removed before the autoinjector is used.

A product container 13 in the form of a syringe is accommodated in the housing 2 so that it cannot be moved along the longitudinal axis L in relation to the housing 2—apart from the mounting of the autoinjector. The product container 13 has a sleeve-shaped syringe body that surrounds a plunger 13b, which is in sealing contact with the inner circumference of the syringe body. At its distal end, the syringe body has an injection needle 13a which is non-detachably connected to the syringe body and whose distal end is formed by the needle tip. A liquid product, for instance a drug, is arranged between the injection needle 13a and the plunger 13b within the syringe body, wherein the liquid product is dispensed from the product container 13 through the hollow injection needle 13a by displacement of the plunger 13b in a dispensing direction, i.e. in a distal direction or towards the injection needle 13a. At its proximal end, the syringe body has a so-called finger flange, which projects radially outwards beyond the outer circumference of the cylindrical syringe body.

The product container 13 is received in a product container holder, referred to as the syringe holder 1, such that it is secured at least against movement along the longitudinal axis L in the distal direction relative to the syringe holder 1. The syringe holder 1 is, as can best be seen from FIG. 2a, positively connected, for instance latched, to the housing 2. For this purpose, the housing 2 has recesses in which latching members engage, the members being formed here at the proximal end of the syringe holder 1. The syringe holder 1 has at least one inwardly projecting shoulder 1b on which a tapered portion of the product container 13, distal to the cylindrical syringe body portion which guides the plunger 13b, is supported.

In order to prevent the product container 13 from being displaceable relative to the syringe holder 1 in the proximal direction, the product container 13 is pressed into engagement with the shoulder 1b at its proximal end by a holder acting on the syringe body. The holder is formed by a retaining spring portion 5c of a mechanism holder 5. The mechanism holder 5 is arranged in relation to the housing 2 along the longitudinal axis L, for instance in a non-displaceable and/or non-rotatable manner The sleeve-shaped mechanism holder 5 can be snapped to the housing 2. The retaining spring portion 5c can compensate for differences in length of the product container 13 that may arise due to manufacturing tolerances, wherein the tight fit of the product container 13 on the shoulder 1b is ensured.

The product container 13 is arranged in relation to the housing 2 such that the needle tip protrudes distally beyond the distal end of the housing 2. In the initial or delivery state (FIGS. 2a-2c) of the autoinjector, i.e. when the pull-off cap 4 is arranged on the autoinjector, the needle 13a is covered by a needle protection cap 14, which in the example shown is designed as a so-called rigid needle shield known to the person skilled in the art, alternatively as a soft needle shield, in order to protect the needle 13a from contamination or to keep the needle 13a and the drug sterile. The rigid needle shield 14 is arranged on a needle holding portion of the syringe body, wherein the tapered portion of the syringe body is located between the needle holding portion and the cylindrical portion of the syringe body. The shoulder 1b is arranged between the syringe body and the proximal end of the rigid needle shield 14, such that a gap, albeit small, is created between the rigid needle shield 14 and the shoulder 1b in order to prevent the shoulder 1b from exerting a force on the rigid needle shield 14, which could, for example, jeopardize the sterility of the needle 13a or the liquid product. The pull-off cap 4 is detachably snap-fit to the housing 2 or a needle shielding sleeve 3, wherein this snap-fit connection is released when the pull-off cap 4 is removed from the housing 2 or the needle shielding sleeve 3. The pull-off cap 4 has a snap hook 4a that engages in a gap between the syringe body, such as its tapered area, and the proximal end of the rigid needle shield 14. When the pull-off cap 4 is removed from the autoinjector, the snap hook 4a hooks into the proximal end of the rigid needle shield 14, releasing the rigid needle shield 14 from the product container 13 and removing it from the autoinjector together with the cover cap 4. Alternatively, the snap hook 4a can hook into a shell surface of the rigid needle shield 14 or into a shell surface of the soft needle shield.

The autoinjector has a needle shielding sleeve 3, which is movable relative to the housing 2 and along the longitudinal axis L by an actuating stroke H_{B} in the proximal direction into an actuated position in order to trigger discharge of the product. In the initial position of the needle protection sleeve 3, as shown in FIGS. 2a-2c, wherein the pull-off cap 4 is removed, the distal end of the needle protection sleeve 3 protrudes distally over the needle tip of the needle 13a, so that access to the needle tip is initially prevented. By displacing the needle protection sleeve 3 by the actuating stroke H_{B}, the needle protection sleeve 3 is displaced in the proximal direction until the needle 13a protrudes from the distal end of the needle protection sleeve 3, such as with a length that may correspond to the injection depth of the needle into the puncture site. The needle 13a should protrude beyond the distal end of the needle protection sleeve 3 to such an extent that a subcutaneous injection can be made. For instance, the housing 2 can form an actuating stop 2b against which the needle protection sleeve 3 rests in the actuated position.

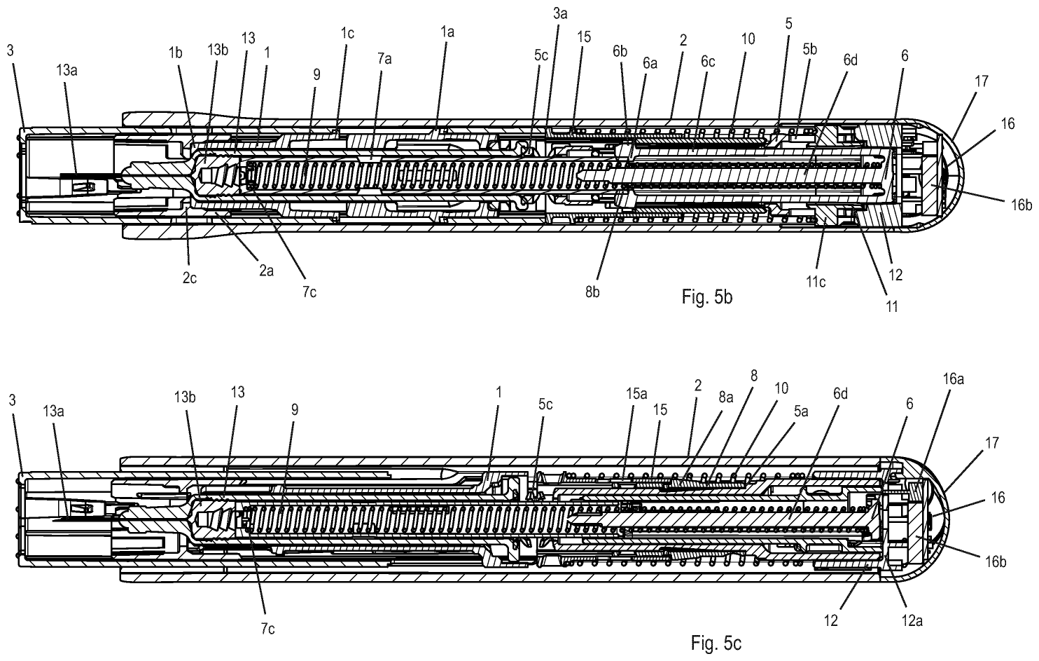

After the injection has been completed, the needle protection sleeve 3 can be displaced relative to the housing 2 from the actuated position along the longitudinal axis L by a needle protection stroke H_{N} in the distal direction to a needle protection position (FIGS. 5a, 5b, 5c). In the needle protection position, the distal end of the needle protection sleeve 3 protrudes distally over the needle tip, preventing access to the needle tip and reducing the risk of injury. As will be described further below, the needle shielding sleeve 3 can be blocked against pushing back out again from the needle shielding position.

The syringe holder 1 has a relief 1a, which points radially outwards, wherein the relief 1a engages in a slot-shaped recess of the needle protection sleeve 3, which is arranged between the housing 2 and the Syringe holder 1. In the initial position of the needle protection sleeve 3 (FIGS. 2a, 2b, 2c) and/or in the needle protection position of the needle protection sleeve 3 (FIGS. 5a-5c), the needle protection sleeve 3, such as the proximal end of the slot-shaped recess, rests against the removal 1a, which prevents movement of the needle protection sleeve 3 in the distal direction. A cam 1c, which is resiliently arranged on the syringe holder 1 and formed by the syringe holder 1, can engage in this slot-shaped recess, or alternatively in another recess of the needle protection sleeve 3. The cam 1c is designed such that during an experiment to displace the needle protection sleeve 3 from the starting position to the actuated position, the cam 1c initially prevents the displacement of the needle protection sleeve 3, wherein the cam 1c is pushed out when the force applied to the needle protection sleeve 3 to push it back exceeds a certain threshold value, whereby the needle protection sleeve 3 is abruptly pushed back to the actuated position. This allows the needle 13a to be inserted suddenly into the puncture site. To pierce the needle 13a or to displace the needle protection sleeve 3 into the actuated position, the distal end of the needle protection sleeve 3 is placed in the piercing point, wherein the housing 2 is then pressed in the direction of the piercing point, whereby when the pressing force exceeds the above-mentioned threshold value, the housing 2 is abruptly displaced towards the piercing point and the needle protection sleeve 3 is displaced relative to the housing 2 into the actuated position.

The housing 2 has a cylindrical holding portion 2a which surrounds the distal end of the syringe holder 1, for instance in a cylindrical shape, and bears against it, whereby the at least one shoulder 1b is held in engagement with the tapered area of the syringe body. Further, in the area of the holding portion 2a, the housing 2 has a translational stop in the form of a holding shoulder 2c, which prevents the syringe holder 1 from being displaceable relative to the housing 2 in the distal direction when the syringe holder 1 rests against the holding shoulder 2c. This is also advantageous for the variants described. Further, the cylindrical holding portion 2a may have a groove 2d which, in connection with a rail 3c attached to the inside of the needle protection sleeve 3, forms an anti-rotation lock for the needle protection sleeve 3. Several grooves 2d and several rails 3c can also be provided.

The autoinjector further has a sleeve-shaped propulsion element 7, which at its distal end has a rib 7c projecting inwards and in the longitudinal direction, on which a first spring 9, which can also be referred to as a pouring spring, is supported. The first spring 9 is arranged within the sleeve-shaped drive element 7. The length of the rib 7c is designed such that the installation space for the first spring 9, which is a helical spring acting as a pressure spring, is reduced and thus the spring 9 in the initial or delivery state (FIGS. 2a-2c) of the autoinjector is preloaded with so much energy that it can pour the product contained in the product container 13 out of the product container 13, for instance completely by displacement of the propulsion element 7 by a pouring stroke H_{A}. In addition, the rib 7c forms a reinforcement of the base of the propulsion element 7 so that the distal area of the propulsion element 7 does not perforate due to the high forces exerted by the extension spring 9. The length of the rib 7c can be different in different autoinjectors, wherein the corresponding length of the rib 7c of the propulsion element 7 can serve to adjust the spring tension of the first spring, which is identical in the different autoinjectors. For example, longer designed ribs can generate a higher spring preload to distribute a higher viscosity liquid. In the as-delivered state (FIGS. 2a-2c) of the device, there is a distance between the piston 13b and the distal end of the propulsion element 7, so that the propulsion element 7 only strikes the piston 13b during the execution of the discharge stroke H_{A} and entrains it in the direction of discharge.

The first spring 9 braces at its proximal end against a holding element 6 which in this example has two arms 6c, wherein a first engagement element 6a and a second engagement element 6b is arranged on each arm 6c. The first engagement element 6a points radially toward the longitudinal axis L, wherein the second engagement element 6b points radially away from the longitudinal axis L. The first engagement element 6a engages in a first recess 7a, which is formed by the drive element 7, thereby preventing movement of the drive element 7 relative to the holding element 6 in the distal direction or in the discharge direction. As a result, the first spring 9 is held in its tensioned or biased state. The holding element 6 has a guide pin 6d which is inserted through the proximal end of the first spring 9 into the core of the spring 9. The guide pin 6d prevents lateral buckling of the first spring 9 during and at the end of the ejection stroke H_{A} of the propulsion element 7.

The autoinjector has a switching module 8, 15 which has a switching sleeve 15 and a locking sleeve 8 surrounded by the switching sleeve 15. In the as-delivered state (FIGS. 2a-2c) of the device, the first engagement element 6a is held in engagement with the first recess 7a by the inner circumference of the locking sleeve 8, which is in contact with the second engagement element 6b.

The switching sleeve 15 is connected to the proximal end 3a of the needle shielding sleeve 3 or at least rests against the proximal end 3a of the needle shielding sleeve 3. A second spring 10, within which the first spring 9 is arranged and which can at least partially surround the switching sleeve 15 and the locking sleeve 8, is supported by its distal end on the switching sleeve 15. A part of the switching sleeve 15 is thus arranged between the needle shielding sleeve 3 and the distal end of the second spring 10. The second spring 10 is a spring made of metal which acts as a compression spring and is designed as a helical spring. The proximal end of the second spring 10 braces against a signal member 11, for instance against a projection 11c, which engages in an axially movable and rotationally fixed manner in the housing 2 and which extends through a slot-shaped groove 5b of the mechanism holder 5. The second spring 10 thus also surrounds the mechanism holder 4 at least partially or completely.

The switching element 15 has a recess 15a in which a locking element 8a of the locking sleeve 8 engages. The locking member 8a is sawtooth-shaped and projects radially away from the longitudinal axis L. The locking member 8a is resiliently arranged on an arm which is formed by the locking sleeve 8. By moving the switching sleeve 15 in the proximal direction, the locking sleeve 8 is entrained in the proximal direction by the engagement of the locking member 8a.

By displacing the needle protection sleeve 3 into the actuated position, the actuating sleeve 15 is also moved by the actuating stroke H_{B}, whereby the second spring 10 is tensioned or biased. If the needle shielding sleeve 3 is not moved completely into the actuated position, the second spring 10 can move the switching sleeve 15 and the needle shielding sleeve 3 back into the initial position, wherein the locking sleeve 8 is also entrained by the switching sleeve 15 via the engagement of the locking member 8a.

The sleeve-shaped signal element 11 is in axially fixed engagement with the propulsion element 7 in the as-delivered state (FIGS. 2a-2c) or before the product discharge is triggered. The signal member 11 has a first engagement member 11a, which engages in a recess 7b of the drive element 7, and a second engagement member 11b. The first engagement member 11a and the second engagement member 11b are resiliently arranged on the end of an arm 11d. The signal member 11 has two such arms 11d with a first engagement member 11a and a second engagement member 11b. The first engagement member 11a points radially toward the longitudinal axis L, whereas the second engagement member 11b points radially away from the longitudinal axis L. In the as-delivered state (FIGS. 2a-2c), the first engagement element 11a is held by the inner circumference of the locking sleeve 8 in the axially fixed engagement with the propulsion element 7. In alternative embodiments, the recess 7b, for instance a recess 7b extending in the longitudinal direction, can be designed in such a way that, during a first partial stroke of the discharge stroke, an axial relative movement takes place between the drive element 7 and the signal member 11 and, during a second partial stroke of the discharge stroke, the first engagement member 11a is held in axially fixed engagement, at least in the distal direction, with the drive element 7. The second engagement member 11b rests against the inner circumference of the switching sleeve 8. The sealing cap 12 has a signal stop 12a against which the signal element 11 can strike to generate a signal and against which the signal element 11 may bear in the as-delivered state (FIGS. 2a-2c) of the device.

To administer the product from the product container 13, the pull-off cap 4 is removed from the autoinjector together with the rigid needle shield 14. The distal end of the needle shielding sleeve 3 is placed at the puncture site of a patient, wherein the housing 2 is moved toward the puncture site, whereby the needle shielding sleeve 3 moves from its initial position into the actuated position by the actuating stroke H_{B} in the proximal direction relative to the housing 2. As a result, the second spring 10 is tensioned or biased, wherein the switching sleeve 15 is entrained or slaved by the needle shielding sleeve 3 by the actuating stroke H_{B}. The locking sleeve 8 has a first recess 8b, which is brought to the position of the second engagement element 6b by displacement of the locking sleeve 8 by the actuating stroke H_{B} along the longitudinal axis L. As a result, the first engagement element 6a is moved out of engagement with the drive element 7 with a movement transverse to and away from the longitudinal axis L, wherein at the same time the second engagement element 6b is moved into engagement with the locking sleeve 8, for instance the first recess 8b thereof. This releases the propulsion element 7 for movement by the discharge stroke H_{A} in the direction of discharge.

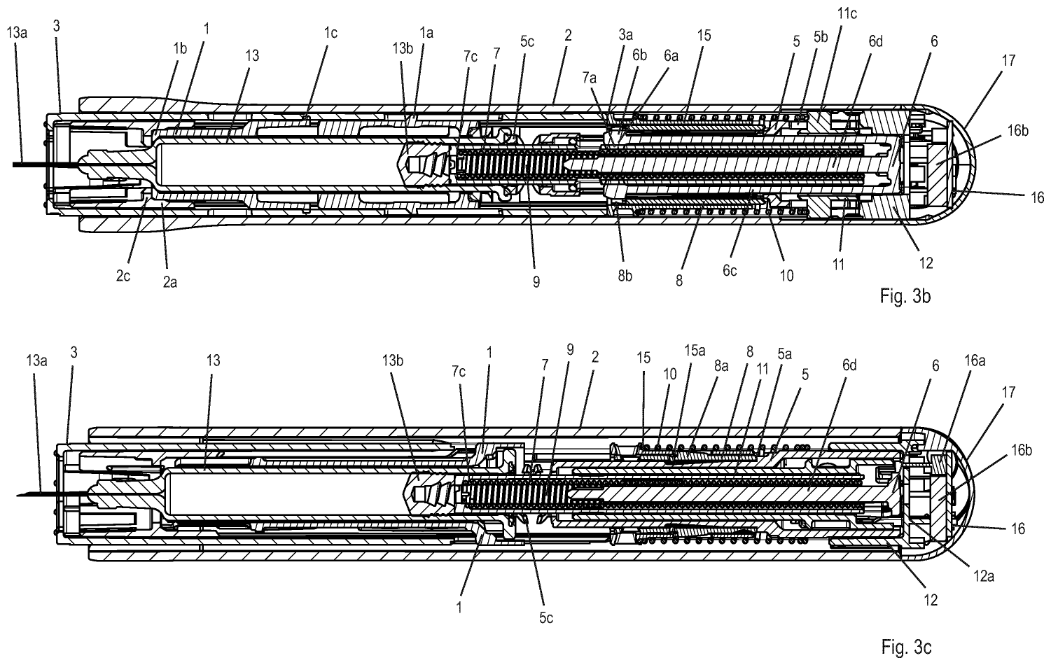

Since the axially fixed coupling between the propulsion element 7 and the holding element 6 is now canceled, the holding element 6, which is movable at least a part relative to the housing 2 and along the longitudinal axis L, can be moved in the proximal direction by the first spring 9, wherein the holding element 6, via the engagement of the second engagement element 6b in the recess 8b, entrains the locking sleeve 8 by a start signal stroke H_{K}, whereby the locking sleeve 8 strikes against a start signal stop 5a, which is formed by the mechanism holder 5, and thereby emits an acoustic and/or tactile signal, which signals to the user of the device that the product dispensing has been started (FIGS. 3a, 3b, 3c). In alternative embodiments, the locking sleeve 8 and/or the start signal stop 5a can be designed in such a manner that no acoustic and/or tactile signal is generated. As a result of the movement of the locking sleeve 8 by the actuating stroke H_{B}, the locking member 8a is enabled for a movement transversely and toward the longitudinal axis L because the mechanism holder 5 has a depression 5d, which allows such a movement of the locking member 8a when the locking sleeve 8 has been moved by the actuating stroke H_{B} or when the needle shielding sleeve 3 is in its actuated position.

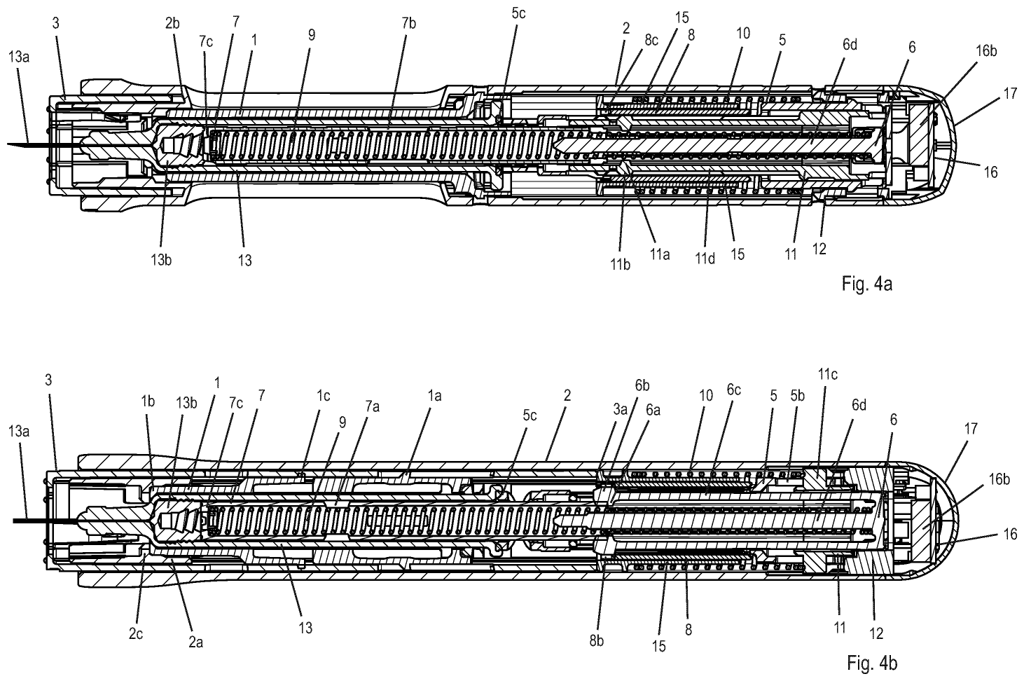

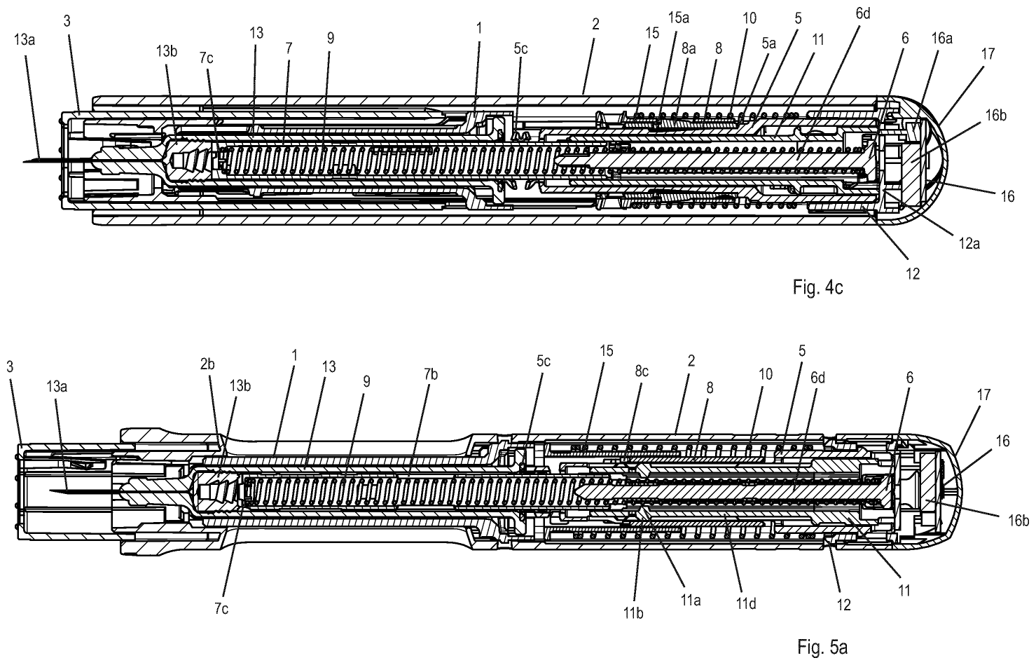

Since the signal element 11 is still axially connected to the propulsion element 7, it is moved in the direction of discharge by a first partial stroke H_{S} of the discharge stroke H_{A}, wherein the signal element 11 is moved away from the signal stop 12a by approximately the first partial stroke H_{S}. At the end of the first partial stroke H_{S}, during which the first and second engagement members 11a, 11b are moved relative to the locking sleeve 8, the first engagement member 11a is pressed out of engagement with the drive element 7, wherein at the same time the second engagement member 11b is moved into the second recess 8c of the locking sleeve 8 with a movement transverse to the longitudinal axis L and radially away from the longitudinal axis L. As a result, the signal member 11 is prevented from moving in the proximal direction relative to the housing 2 or the locking sleeve 8. The second engagement element 11b is held in engagement with the second recess 8c by the outer circumference of the propulsion element 7 when the propulsion element 7 is moved by its second partial stroke of the discharge stroke H_{A}. The outer circumferential surface of the drive element 7 holds the second engagement element 6b in engagement with the first recess 8b of the locking sleeve 8, as can best be seen from FIG. 4b. At the end of the discharge stroke H_{A}, the propulsion element 7 releases the second engagement element 11b from engagement with the locking sleeve 8, whereby the second engagement element 11b is moved out of engagement with the recess 8c, for instance towards the longitudinal axis L, so that the second spring 10 accelerates the signal element 11 against the direction of discharge, i.e. in the proximal direction, so that an acoustic and/or tactile signal is generated when the signal element 11 strikes the signal stop 12a (FIGS. 4a-4c). In alternative embodiments, the signal element 11 and/or the signal stop 12a may be designed such that no acoustic and/or tactile signal is generated.

As can best be seen from FIG. 4b, the second engagement element 6b remains engaged in the first recess 8b, preventing movement of the locking sleeve 8 in the distal direction relative to the housing 2.

By removing the autoinjector from the injection site, the second spring 10 can move the switching sleeve 15 and the needle protection sleeve 3 from the actuated position to the needle protection position (FIGS. 5a-5c) by the needle protection stroke H_{N}, wherein the locking element 8a is pushed out of engagement with the recess 15a, the switching sleeve 15 moving in the distal direction relative to the locking sleeve 8. When the needle protection sleeve 3 is in its needle protection position, the locking element 8a snaps together with the switching sleeve 15, for instance at a proximal edge of the switching sleeve 15 or alternatively in another recess of the switching sleeve 15, wherein the locking element 8a prevents the needle protection sleeve 3 from being pushed back into its actuated position. In the attempt to push the needle shielding sleeve 3 back from the needle shielding position into the actuated position, the switching device 15 hits against the locking member 8a, which prevents the movement of the needle shielding sleeve 3 into the actuated position. For this purpose, the locking sleeve 8 braces axially against the start signal stop 5a of the mechanism holder 5.

The electronic module 16 with a sensor 16a is configured to measure the axial movement of the signal element 11 from a position at the start of the discharge, wherein the signal element 11 is entrained in the direction of discharge, to a position at the end of the discharge, wherein the signal element 11 strikes the signal stop 12a.

By providing the signal element 11, which can be moved axially relative to the housing 2, the injection can be carried out directly by monitoring the positions, for instance the position at the start of the distribution and the position at the end of the distribution of the signal element 11.

This monitoring of the positions of the signal element via the electronic module can be considered as a supplement or as an alternative to the acoustic and/or tactile signal generation already mentioned above in order to improve the patient's therapy.

The sensor 16a of the electronic module 16 is designed as a switching detector to detect or detect the positional displacement of the signal element 11. Further, the autoinjector comprises a switching actuator 18 to actuate the sensor 16a, for instance the switching detector. In alternative embodiments, the switching actuator 18 may be provided on the signal element 11 or the signal element 11 may be designed as a switching actuator 18. The switching actuator 18 can suitably deflect or redirect a movement of the signal element 11 to be detected and can be designed in one or more parts for this purpose. In addition to a ram mounted so that it can move in an axial direction, the switching actuator can also comprise a hinge or a gearbox to mediate the movement of the signal element to the detector.

As an alternative or in addition to the movement or position of the signal element 11, the position or movement of the holding element 6 can also be detected. When triggered, the first spring 9 moves this at least part of the way relative to the housing 2 and along the longitudinal axis L in the proximal direction, whereby one or the sensor 16a is also actuated by means of the switching actuator 18, or by means of a further switching actuator 19.

Further, the processor 16c is coupled and configured with the sensor 16a and with the battery 16b of the electronic module 16 such that the start and/or the end of the discharge of the autoinjector can be registered or sensed by means of sensor 16a. The axial movement of the signal element 11 from a position at the start of the distribution to a position at the end of the distribution is recorded or detected.

The processor 16c comprises evaluation electronics configured to identify processes in or states of the injection device during an injection process based on measurements of the sensor 16a.

The processor 16c may be configured to operate a timer. The processor 16c of the electronic module 16 may be configured to register or sense the elapsed time since the last distribution and/or to register or sense after a predetermined duration after the registered or sensed end of the distribution and/or to register or sense after the registered or sensed start of the distribution.

After the detection or detection of the start position of the signal element 11 and/or the end position of the signal element 11, or alternatively after a fixed duration after the registered or sensed end and/or after the registered or sensed start of the distribution, the light source 16d can be activated.

The light source 16d is accommodated in a light source housing 10. This housing can be used for protection and better display.

The light source 16d displays information on the state of the autoinjector. The light source 16d indicates the start and/or end of the autoinjector discharge. Further, the expiry of a holding time or waiting time can be displayed additionally or alternatively, wherein the patient can remove the autoinjector from the skin after this time has elapsed.

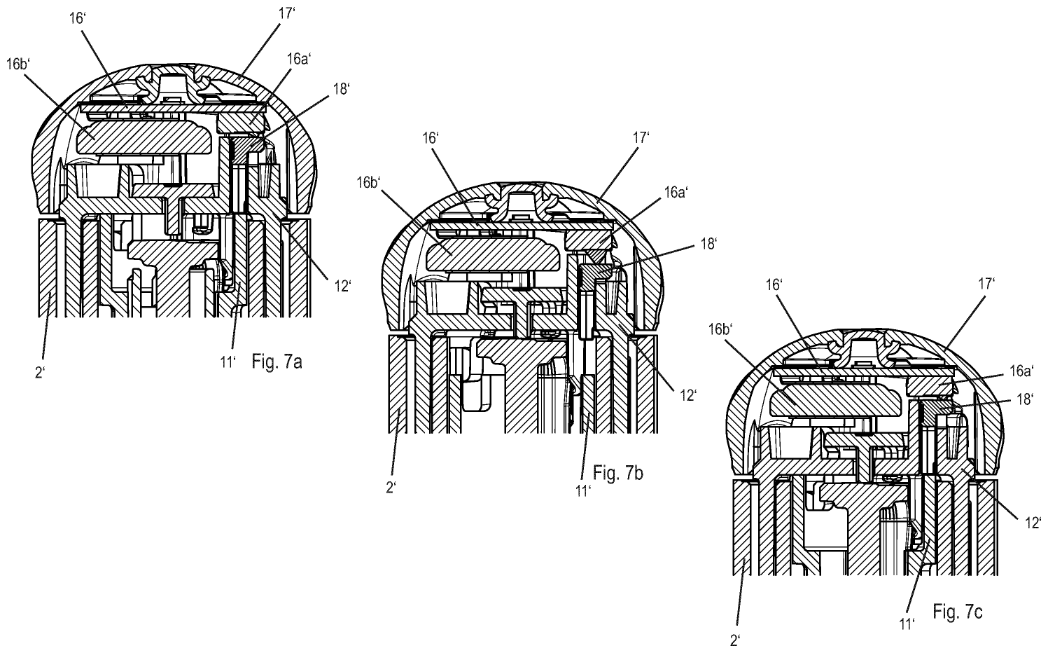

FIGS. 7a-7c show a different embodiment of the invention from the embodiments already disclosed above, wherein only the proximal end of the autoinjector is apparent. This embodiment differs from the embodiments already disclosed in the structure and function of the electronic module.

The sensor 16a′ of the electronic module 16′ is designed as a switching detector to detect or detect the positional displacement of the signal element 11′. Further, the autoinjector comprises a switching actuator 18′ to actuate the sensor 16a′, for instance the switching detector. The sensor 16a′ is arranged axially and rotationally fixed in an electronic module housing 17′ of the electronic module 16′ or alternatively in a housing 2′ of the autoinjector. The switching actuator 18′ has a preload force, for instance a spring force, or is designed to be preloaded, such as resilient. The switching actuator 18′ may be designed in the shape of a pin. The switching actuator 18′ is axially movable, displaceable or slidable in a sealing cap 12′ of the autoinjector or alternatively can be arranged in the housing 2′ of the autoinjector. At the start of the release, the signal element moves in the distal direction. Due to the released spring force and/or gravity, the switching actuator 18′ also moves in the distal direction

Alternatively or cumulatively to the force of gravity, this force can cause the switching actuator 18′ to be moved or to move in the distal direction during the movement of the signal element 11′ in the distal direction, as can be seen in FIG. 7b. At the end of the distribution, the signal element 11′ is moved back to the proximal position, wherein the signal element 11′ takes the switching actuator 18′ with it and is also moved to the proximal position, as represented in FIG. 7c. The axial movement of the signal element 11′ in the distal direction at the start of the distribution and the axial movement of the signal element 11′ in the proximal direction at the end of the distribution can thus be transmitted from the switching actuator 18′ to the sensor 16a′, for instance to the switching detector. The sensor 16a′, for instance the switching detector, is configured to measure the axial movement of the switching actuator 18′ from a position at the start of the discharge, wherein the switching actuator 18′ is movable or displaceable in the discharge direction, to a position at the end of the discharge, wherein the switching actuator 18′ is movable or displaceable in the proximal direction. The sensor 16a′, particularly the switching detector, can thus detect or detect the position of the signal element 11′.

### LIST OF REFERENCE SIGNS

1 Syringe holder
1a Protrusion
1b Shoulder
1c Cam
2 Housing
2′ Housing
2a Holding portion
2b Actuation stop
2c Holding shoulder
2d Groove
3 Needle protection sleeve
3a Proximal end
3b Rail
4 Pull-off cap
4a Snap-in hook
5 Mechanism holder
5a Start signal stop
5b Groove
5c Retaining spring portion
5d Indentation
6 Holding element
6a First engagement element
6b Second engagement element
6c Arm
6d Guide pin
7 Drive element
7a First recess
7b Second recess
7c Rib
8 Locking sleeve
8a Locking member
8b First recess
8c Second recess
9 First spring/discharge spring
10 Second spring/needle shielding spring
11 Signal member
11′ Signal member
11 a First engagement member
11b Second engagement member
11c Protrusion
11d Arm
12 Closure cap
12′ Closure cap
12a Signal stop
13 Product container/syringe
13a Needle
13b Piston
14 Rigid needle shield/needle shielding cap
15 Switching sleeve
15a Recess
16 Electronics module
16′ Electronics module
16a Sensor
16a′ Switch detector
16b Battery
16b′ Battery
16c Processor
16d Light source/LED
17 Electronics module housing
17′ Electronics module housing
18, 19 Switch actuator
18′ Switch actuator
10 Light source/LED housing
L Longitudinal axis

What is claimed is:

1. An autoinjector for dispensing a liquid product, such as a drug, comprising:
a) a housing comprising a product container arranged therein, the product container comprising a piston displaceable in a dispensing direction for dispensing the liquid product contained in the product container;
b) a propulsion element configured to act on the piston during product dispensing, and a first spring configured to act on the propulsion element;
c) a signal element, a signal stop and a second spring, which exerts a spring force on the signal element counter to the dispensing direction, wherein the signal element is in an axially fixed coupling with the propulsion element such that the signal element is slaved and the second spring is biased during a displacement of the propulsion element in the dispensing direction, wherein the axially fixed coupling between the signal element and the propulsion element is releasable, and the signal element is configured to be accelerated counter to the dispensing direction and relative to the propulsion element and/or the housing by means of the second spring, wherein upon the signal element being released from the axially fixed coupling with the propulsion element and accelerated by the second spring, the signal element strikes against the signal stop; and
d) an electronic module with a sensor configured to measure an axial movement of the signal element from a position at a start of the product dispensing, wherein the signal element is slaved in the dispensing direction, to a position at an end of the product dispensing, wherein the signal element strikes the signal stop.

2. The autoinjector according to claim 1, wherein the electronic module is a separate electronic unit and is configured to be connected to the housing of the autoinjector.

3. The autoinjector according to claim 2, wherein the electronic module is connected to the housing of the autoinjector by means of a bayonet lock.

4. The autoinjector according to claim 1, wherein the electronic module comprises an energy source and a processor, wherein the processor and the energy source are coupled to the sensor such that the start and the end of the product dispensing of the autoinjector are sensed by the sensor.

5. The autoinjector according to claim 4, wherein the electronic module comprises a timer configured to measure a duration of the product dispensing of the autoinjector.

6. The autoinjector according to claim 4, wherein the electronic module comprises a wireless communication unit for communicating with an external electronic apparatus and/or a status indicator for indicating at least one position of the signal element.

7. The autoinjector according to claims 5, wherein a status indicator is activated after a fixed duration after the sensed end and/or after the sensed start of the product dispensing.

8. The autoinjector according to claim 1, wherein the sensor is configured as a switching detector to sense the start and the end of the product dispensing of the autoinjector.

9. The autoinjector according to claim 8, wherein the autoinjector comprises a switching actuator to actuate a switch or button of the switching detector.

10. The autoinjector according to claim 1, wherein the signal element comprises a first engagement element configured to releasably engage in the propulsion element such that the propulsion element is coupled axially fixedly to the signal element, wherein the axially fixed coupling between the propulsion element and the signal element is released when the signal element is out of the engagement with the propulsion element.

11. The autoinjector according to claim 1, wherein the signal stop is formed by the housing or by an element which is connected to the housing at least in an axially fixed manner, the signal stop being arranged along a longitudinal axis (L) of the housing and in an alignment with the signal element.

12. The autoinjector according to claim 11, wherein the signal stop is formed by the housing or by the element which is connected to the housing further in a rotationally fixed manner

13. The autoinjector according to claim 12, wherein the signal stop is formed by a sealing cap configured to close a proximal end of the housing.

14. The autoinjector according to claim 1, wherein a needle protection sleeve configured to act on the second spring is displaceable from an initial position relative to the housing along a longitudinal axis of the autoinjector in a proximal direction, in an actuating stroke for triggering the product dispensing, whereby the second spring is biased and the product dispensing is triggered.

15. The autoinjector according to claim 14, further comprising a switching module arranged kinematically or/and geometrically between the second spring and the needle protection sleeve, wherein the switching module is slaved by the needle protection sleeve in the proximal direction when the needle protection sleeve is displaced from a starting position in the proximal direction.

16. The autoinjector according to claim 14, wherein the signal element comprises a second engagement element, which can be moved out of the propulsion element into an axially fixed engagement with the needle protection sleeve or a switching module by movement of a first engagement element, wherein the first engagement element and the second engagement element cooperate with one another such that the second engagement element engages axially fixedly in an engagement with the needle protection sleeve or the switching module prior to the first engagement element being disengaged from an engagement with the propulsion element.

17. The autoinjector according to claim 16, wherein the propulsion element is movable in a distal direction relative to the signal element by means of the first spring when the first engagement element is out of the engagement with the propulsion element and the second engagement element is in the engagement with the needle protection sleeve or the switching module.

18. The autoinjector according to claim 16, wherein the propulsion element prevents the second engagement element from moving out of the axially fixed engagement with the needle protection sleeve or the switching module when the propulsion element moves in a distal direction relative to the signal element, wherein the propulsion element at the end of a product dispensing stroke allows the second engagement element to disengage from the engagement with the needle protection sleeve or the switching module, whereby the signal element is accelerated by the second spring counter to the dispensing direction and strikes against the signal stop.
